(19) United States
(12) Patent Application Publication
(10) Pub. No.: US 2016/0015913 A1
(43) Pub. Date: Jan. 21, 2016

(54) CERVICAL INSPECTION DEVICE AND METHOD FOR CERVICAL INSPECTION

(71) Applicant: MARK NEUMAN, Queens, NY (US)

(72) Inventor: MARK NEUMAN, Queens, NY (US)

(21) Appl. No.: 14/554,861

(22) Filed: Nov. 26, 2014

Related U.S. Application Data
(60) Provisional application No. 62/026,386, filed on Jul. 18, 2014.

(51) Int. Cl. A61M 13/00; A61B 5/107; A61B 1/303; A61B 5/00; A61B 1/05; A61B 1/00

(52) U.S. Cl. CPC A61M 13/003; A61B 1/05; A61B 1/00154; A61B 1/303; A61B 1/00045; A61B 5/435; A61B 5/1076; A61B 5/1079; A61B 1/00016; A61M 2210/1475

(57) ABSTRACT

A cervical inspection device includes a sealing body defined by an outer perimeter and being constructed for sealing placement about the vaginal introitus of a patient, the sealing body having an opening for introducing gas under pressure into the vaginal vault of the patient; and an extendable member connected to the sealing body in a region radially inwardly relative to the outer perimeter of the sealing body and being extendable in a direction transverse to the perimeter of the sealing body to form a generally tubular sheath.

### CROSS-REFERENCES TO RELATED APPLICATIONS

This application claims the benefit of prior filed U.S. provisional Application No. 62/026,386, filed Jul. 18, 2014, pursuant to 35 U.S.C. 119(e), the disclosure of which is incorporated herein by reference.

### BACKGROUND OF THE INVENTION

The present invention relates to a cervical inspection device and a method for cervical inspection.

The following discussion of related art is provided to assist the reader in understanding the advantages of the invention, and is not to be construed as an admission that this related art is prior art to this invention.

Currently, when a pregnant female presents to her Obstetrician's office or to the Labor & Delivery department of a hospital, a bimanual exam is performed to determine cervical dilation and effacement. The exam subsists of the physician inserting two fingers into the vagina, locating the cervix, and determining the consistency of the cervix at that time, oftentimes requiring tremendous physical pressure from the examiner as he/she attempts to locate the cervix and determine its current dilation and effacement. This is both an imprecise and subjective examination. Additionally, the exam can be painful and/or cause the patient much anxiety due to the nature of the examination. Furthermore, once a patient has ruptured her membranes, colloquially known as “breaking her water,” Obstetricians check the patient less frequently to avoid introducing infection into the uterus.

It would therefore be desirable and advantageous to provide an apparatus that provides a new and precise method to determine cervical dilation and effacement, while avoiding pain, anxiety, and potential for introducing infection.

### SUMMARY OF THE INVENTION

According to one aspect of the present invention, A cervical inspection device, comprising a sealing body defined by an outer perimeter and being constructed for sealing placement about the vaginal introitus of a patient, said sealing body having an opening for introducing gas under pressure into the vaginal vault of the patient; and an extendable member connected to the sealing body in a region radially inwardly relative to the outer perimeter of the sealing body and being extendable in a direction transverse to the perimeter of the sealing body to form a generally tubular sheath.

The perimeter of the sealing body can be placed about the vaginal introitus of the patient to create a seal around the vaginal introitus. A source of gas under pressure, for example carbon dioxide, can be connected to the opening of the sealing body for example via an appropriate tubing. The gas can then be introduced through the opening of the sealing body to insufflate the vaginal vault, thereby causing separation between the vaginal mucosa and the cervix. Through the sealing body, a fiber optic camera can be introduced into the tubular sheath. The camera can be advanced in the sheath through the vaginal introitus and along the vaginal vault to bring the camera in proximity to the cervix of the patient.

According to another advantageous feature of the invention, the sealing body can have different shapes and can be made of different materials. In principal the sealing body can have any shape and be made of any material so long as it provides sufficient sealing around the vaginal introitus of the patient when placed thereon.

In one advantageous embodiment the sealing body can have a triangular-shaped outline for example in the manner of a female groin protector so as to conform to the anatomical contours surrounding the vaginal introitus. This provides a particularly good sealing effect. Other possible shapes include an oblong or oval shape or a circular shape.

In another embodiment the sealing body can have a convex side and a concave side in the manner of a respiratory mask. Such a shape of the sealing body facilitates handling of the cervical inspection device during use. In this embodiment the user can hold the cervical inspection device on its convex side and place the concave side of the cervical inspection device about the vaginal introitus

In another advantageous embodiment, the sealing body can have a sealing cuff arranged along the outer perimeter. The sealing cuff can be filled with air or a liquid to provide cushioning. This can also be advantageous to facilitate the sealing effect of the sealing body. In another embodiment the sealing cuff may also be inflatable.

Examples of materials of which the sealing body can be made include plastic materials such as polyethylene or polystyrene, rubber, latex and the like.

According to another advantageous feature of the invention, the opening in the cuff can be constructed to receive a catheter. The catheter can be arranged so as to project directly into the vaginal introitus to provide a more direct application of the gas under pressure into the vagina.

According to another advantageous embodiment, the extendable member can also be made of different materials. Examples for materials that the extendable member can be made of are plastic materials or latex.

According to another advantageous embodiment, the extendable member can be constructed as a preformed tubular sheath that is folded prior to insertion of the camera. Upon insertion of the camera through the cuff into the folded sheath the folded sheath then extends corresponding to the degree the camera is inserted into the sheath.

In another embodiment the extendable member can be made of an elastic material. For example, the extendable member can be made of a material such as latex that. In this way for example a camera can be placed against the extendable member when the sealing body is place about the vaginal introitus and the extendable member can then be stretched by pushing the camera in the direction toward the cervix of the patient.

In another embodiment, an end portion of the extendable sheath is configured translucent.

According to another aspect of the invention a system for cervical inspection includes, a cervical inspection device, including a sealing body defined by an outer perimeter and being constructed for sealing placement about the vaginal introitus of a patient, the sealing body having an opening for introducing gas under pressure into the vaginal vault of the patient; and an extendable member connected to the sealing body in a region radially inwardly relative to the outer perimeter of the sealing body and being extendable in a direction transverse to the perimeter of the sealing body to form a generally tubular sheath; a camera for insertion into the tubular sheath to obtain an image of the cervix of the patient; and a monitor connected to the camera to display the image of the cervix of the patient.

According to another aspect of the present invention a method for cervical inspection includes the steps of providing a cervical inspection device, including a sealing body defined by an outer perimeter and being constructed for sealing placement about the vaginal introitus of a patient, said sealing body having an opening for introducing gas under pressure into the vaginal vault of the patient, and an extendable member connected to the sealing body in a region radially inwardly relative to the outer perimeter of the sealing body and being extendable in a direction transverse to the perimeter of the sealing body to form a generally tubular sheath; placing the outer perimeter of the cervical inspection device about the vaginal introitus of the patient, creating a seal about the vaginal introitus; introducing a gas under pressure into the vaginal vault of the patient via the opening of the sealing body, thereby expanding the vaginal vault; inserting a camera into the sheath, and advancing the camera in the vagina of the patient until the cervix is visualized.

After visualizing the cervix the dilation of the cervix can be measured. The image obtained by the camera can be shown on a screen, which can then be printed as an objective finding, quantifying the cervical dilation and effacement.

The measurement can be performed by using a ruler. The ruler can be a stereoscopic ruler, a manual ruler, or a laser based ruler. The ruler may also be provided in a different channel on the scope or integrated into the scope.

According to another advantageous feature of the invention, the measurement can be wirelessly linked to electronic medical records of the patient.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 shows a perspective side view of an embodiment of the cervical inspection device according to the present invention;

FIG. 2 shows a front view of the cervical inspection device of FIG. 1 taken along the direction indicated by the letter A.

FIG. 3 shows a side view of the cervical inspection device during use, place on the vaginal introitus of a patient.

FIG. 4 shows a side view of the cervical inspection device during use, with a fiber optic camera inserted in to the extendable sheath.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way. It should also be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

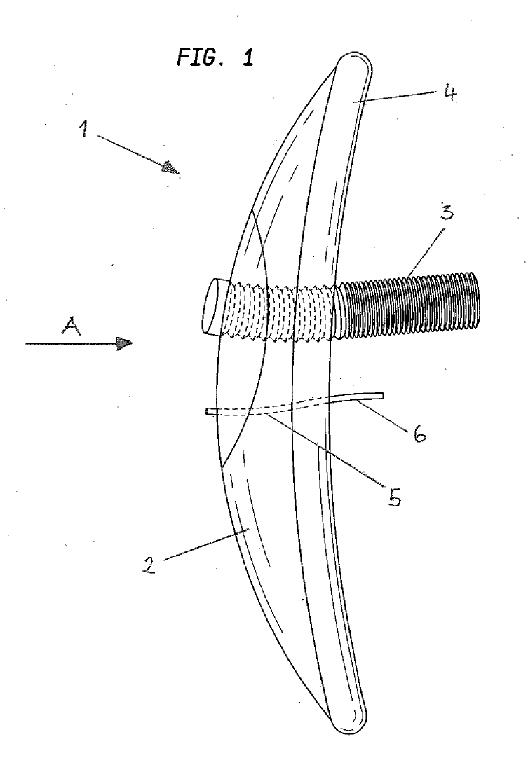

Turning now to the drawing, and in particular to FIG. 1, there is shown a perspective side view of an embodiment of the cervical inspection device 1 according to the present invention. The embodiment of the cervical inspection device 1 shown in FIG. 1 has a sealing body 2, an extendable member 3 and an opening 5 for introducing a gas under pressure for example CO₂ into the vaginal vault of the patient. In this embodiment, the extendable member 3 is constructed as a preformed generally tubular sheath arranged approximately in a center region of the sealing body 2. In FIG. 1 the extendable member is shown in the non-extended state. A catheter 6 can be attached to the opening 5 and extended into the vaginal vault of the patient. The sealing body 2 may have a cuff 4 formed around the outer perimeter of the sealing body 2. It can further be seen from FIG. 1 that the cervical inspection device has a concave side and a convex side.

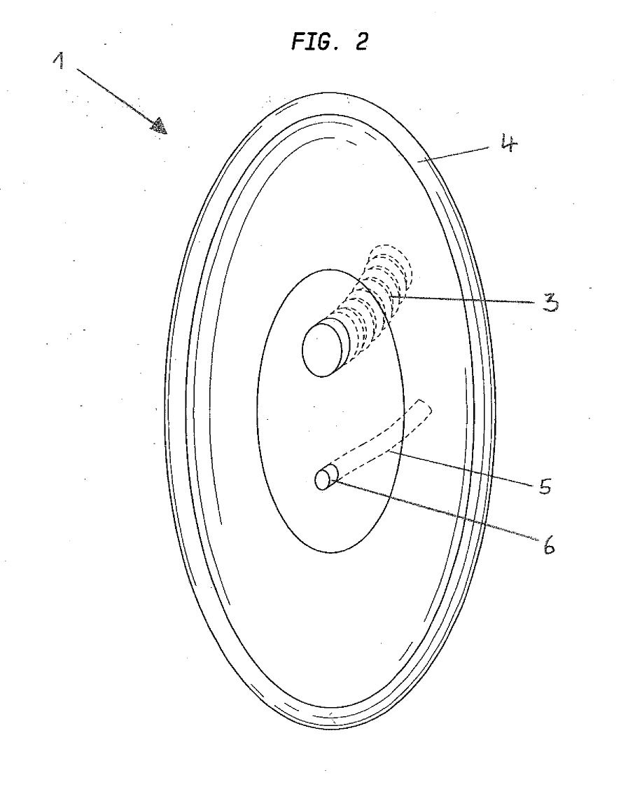

FIG. 2 shows the embodiment of the cervical inspection device 1 of FIG. 1 in a perspective front view taken along the direction indicated by the letter A in FIG. 1. In this particular embodiment, the cervical inspection device has an oval shape.

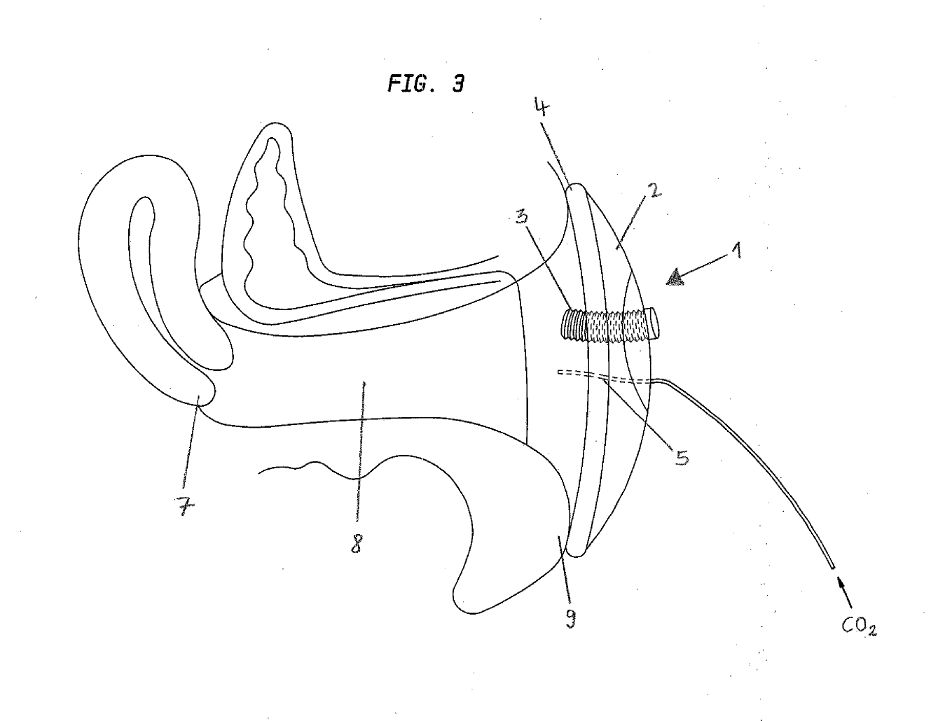

FIG. 3 shows the cervical inspection device 1 during use. The cervical inspection device 1 is placed about the vaginal introitus 9 of the patient, with the extendable member 3 facing toward the cervix 7 of the patient. A gas under pressure, for example CO₂, as indicated by the arrow, can be introduced through the opening 5 into the vaginal vault 8, thereby insufflating the vaginal vault 8.

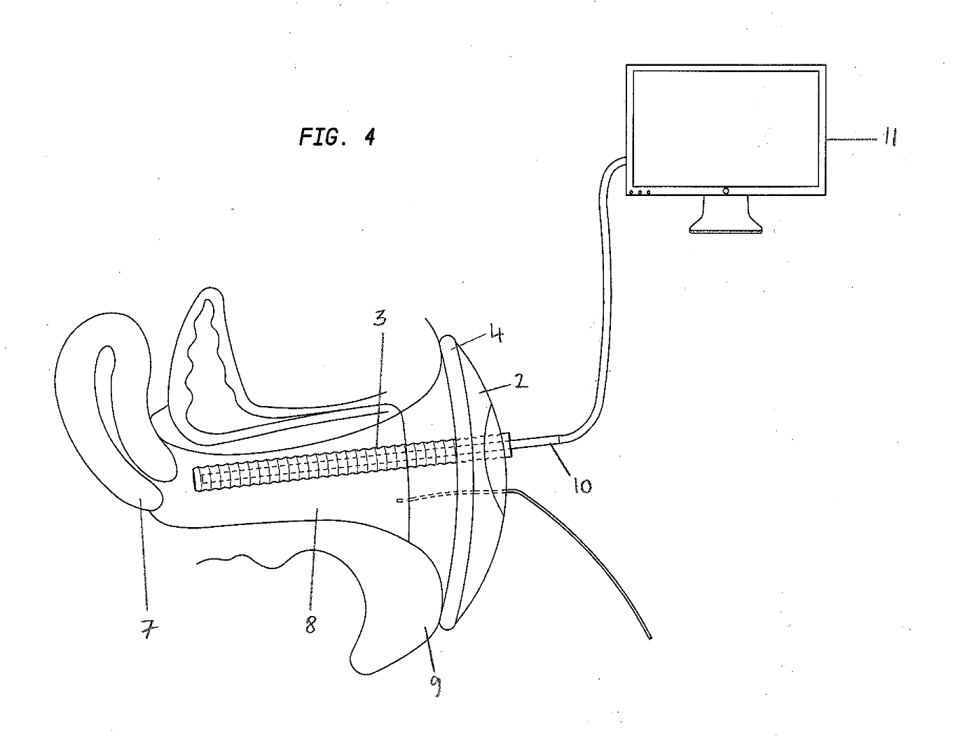

FIG. 4 shows the cervical inspection device as shown in FIG. 3 with the extendable member 3 being extended in the direction toward the cervix 7 of the patient by inserting a fiber optic camera 10 into the extendable member 3 and moving the fiber optic camera 10 in the direction toward the cervix 7. As can be seen in FIG. 4, the fiber optic camera 10 is moved into close proximity of the cervix 7 thereby enabling visualizing the cervix 7. The fiber optic camera 10 is connected to a monitor 11 for displaying the image of the cervix 7 obtained by means of the fiber optic camera 10. The image of the cervix 7 obtained by the camera 10 and displayed on the monitor 11 can then be used to measure the cervix 7.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

What is claimed is:

1. A cervical inspection device, comprising:
a sealing body defined by an outer perimeter and being constructed for sealing placement about the vaginal introitus of a patient, said sealing body having an opening for introducing gas under pressure into the vaginal vault of the patient; and
an extendable member connected to the sealing body in a region radially inwardly relative to the outer perimeter of the sealing body and being extendable in a direction transverse to the perimeter of the sealing body to form a generally tubular sheath.

2. The cervical inspection device of claim 1, wherein a side of the sealing body which faces away from the vaginal introitus during use has a convex shape.

3. The cervical inspection device of claim 1, wherein the sealing body is made of a material selected from the group consisting of a plastic material, rubber, and latex.

4. The device of claim 1, wherein the sealing body has a sealing cuff arranged along the outer perimeter.

5. The device of claim 4, wherein the sealing cuff is constructed inflatable.

6. The device of claim 1, wherein the extendable member is constructed as folded preformed sheath.

7. The device of claim 1, further comprising a valve arranged in the opening for controlling the flow of the gas under pressure.

8. A system for cervical inspection, comprising:
a cervical inspection device, comprising a cuff having an outer perimeter and being constructed for sealing placement about the vaginal introitus of a patient, said cuff having an opening for introducing gas under pressure into the vaginal vault of the patient; and an extendable member sealingly connected to the cuff in a region radially inwardly relative to the outer perimeter of the cuff and being extendable to form a generally tubular sheath;
a camera for insertion into the tubular sheath to obtain an image of the cervix of the patient; and
a monitor connected to the camera to display the image of the cervix of the patient.

9. A method for cervical inspection, comprising:
providing a cervical inspection device, comprising a sealing body defined by an outer perimeter and being constructed for sealing placement about the vaginal introitus of a patient, said sealing body having an opening for introducing gas under pressure into the vaginal vault of the patient, and an extendable member connected to the sealing body in a region radially inwardly relative to the outer perimeter of the sealing body and being extendable in a direction transverse to the perimeter of the sealing body to form a generally tubular sheath;
placing the outer perimeter of the cervical inspection device about the vaginal introitus of the patient, creating a seal about the vaginal introitus;
introducing a gas under pressure into the vaginal vault of the patient via the opening of the sealing body, thereby expanding the vaginal vault;
inserting a camera into the sheath and advancing the camera in the vagina of the patient until the cervix is visualized.

10. The method for claim 9, further comprising measuring a dilation of the cervix.

11. The method for claim 9, further comprising linking the measurement wirelessly to electronic medical records of the patient.
